(19) United States
(12) Patent Application Publication
(10) Pub. No.: US 2006/0035926 A1
(43) Pub. Date: Feb. 16, 2006

(54) Benzothiazolium compounds

(76) Inventors: Shiow-Ju Lee, Tainan (TW); Yung-Ning Yang, Taipei (TW)

Correspondence Address: FISH & RICHARDSON PC, P.O. BOX 1022, MINNEAPOLIS, MN, 55440-1022, US

(21) Appl. No.: 11/204,791

(22) Filed: Aug. 15, 2005

Related U.S. Application Data
(60) Provisional application No. 60/601,390, filed on Aug. 13, 2004.

(51) Int. Cl. A61K 31/47; A61K 31/428

(52) U.S. Cl. 514/310; 514/367

(57) ABSTRACT

This invention relates to a method of inhibiting NO production and TNFα and treating coronaviral infection by administering an effective amount of a compound of the following formula: 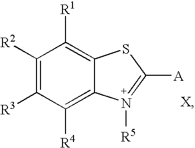
wherein R¹, R², R³, R⁴, R⁵, A, and X are as defined herein.

### RELATED APPLICATIONS

This application claims priority to U.S. Provisional Application No. 60/601,390, filed on Aug. 13, 2004, the contents of which are incorporated herein by reference.

### BACKGROUND

Nitric oxide (NO) is an important pleiotropic molecule mediating a wide range of physiological and pathophysiological processes. Overproduction of NO has been implicated in various pathological processes including septic shock, virus infection, tissue damage following inflammation, cancer, atherosclerosis, and arthritis.

NO is produced from L-arginine and molecular oxygen by three distinct isoforms of nitric oxide synthase (NOS), i.e., neural NOS (nNOS), endothelial NOS (eNOS), and inducible NOS (iNOS). Among the three NOSs, iNOS can be induced by endotoxins or cytokines (e.g., TNF α and IL-6) to produce a high level of NO. Inhibiting expression or activity of iNOS is a major task of preventing and eliminating NO overproduction.

### SUMMARY

This invention is based on a discovery that a group of benzothiazolium compounds, unexpectedly, suppress NO production and possess inhibitory activities against coronavirus.

One aspect of this invention relates to a method of suppressing NO production (via expression of iNOS) or lowering production of TNF α or IL-6 by administering to a subject in need thereof an effective amount of a compound of the following formula: 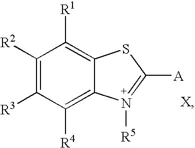
wherein each of R¹, R², R³, and R⁴, independently, is H, halo, alkyl, alkenyl, akynyl, alkoxyl, aryl, heteroaryl, cylcyl, heterocyclyl, nitro, cyano, amino, amido, or alkoxycarbonyl, or R¹, R², and the two carbon atoms to which they are attached, together form a 3-8 membered ring containing 0, 1, 2, or 3 heteroatoms, or R², R³, and the two carbon atoms to which they are attached, together form a 3-8 membered ring containing 0, 1, 2, or 3 heteroatoms, or R³, R⁴, and the two carbon atoms to which they are attached, together form a 3-8 membered ring containing 0, 1, 2, or 3 heteroatoms; R⁵ is H, alkyl, aryl, heteroaryl, cyclyl, or heterocyclyl; A is H, alkyl, aryl, heteroaryl, cyclyl, heterocyclyl, alkoxy, amino, halo, mercapto, thioalkoxy, 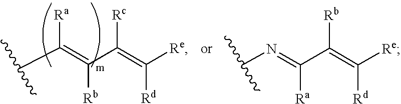
in which m is 0, 1, 2, or 3; each of R^{a}, R^{b}, R^{c}, R^{d} and R^{e} independently is H; alkyl, akenyl, akynyl, halo, aryl, heteroaryl, cyclyl, heterocylyl, alkoxy, mercapto, thioalkoxy, amino, amido, or R^{d}, R^{e}, and the carbon atom to which they are attached, together form a 3-8 membered ring containing 0, 1, 2, or 3 heteroatoms, which is optionally fused with aryl, heteroaryl, cyclyl, or heterocyclyl; optionally A, R⁵, the N atom to which R⁵ is attached, and the carbon atom to which A is attached, together form a 4-8 membered ring containing 1, 2, or 3 heteroatoms; and X is an anion or a negatively charged group attached to an atom of R¹, R², R³, R⁴, R⁵, or A.

Referring to the above formula, one subset of the compounds feature that X is I—, Br⁻, Cl⁻, ClO₄⁻, C₂H₅SO₄⁻, or 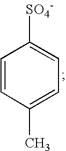
or a negatively charged group attached to an atom of R¹, R², R³, R⁴, R⁵, or A, the negatively charged group being selected from —CO₂⁻, —SO₃⁻, or PO₃²⁻.

Another subset of the compounds feature that A is alkyl or R⁵ is alkyl.

Still another subset of the compounds feature that A is 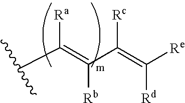
and R⁵ is alkyl. In some of these compounds, one of R^{d} and R^{e} is substituted or unsubstituted phenyl, thioalkyl, amino, or amido. In some other compounds, R^{d}, R^{e}, and the carbon atom to which they are attached, together form a 3-8 membered ring containing 0, 1, or 2 heteroatoms. As an example, A can be 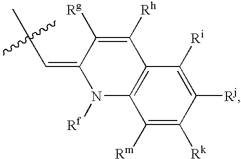
R^{f} being H, alkyl, alkenyl, aryl, cyclyl, heteroaryl, or heterocyclyl; and each of R^{g}, R^{h}, Rⁱ, R^{j}, R^{k}, and R^{m}, independently being H, alkyl, alkenyl, aryl, cyclyl, heteroaryl, heterocyclyl, alkoxyl, halogen, nitro, or cyano. As another example, A can be 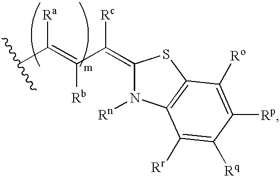
Rⁿ being H, alkyl, alkenyl, aryl, cyclyl, heteroaryl, heterocyclyl, and each of R^{o}, R^{p}, R^{q}, and R^{r}, independently, being H, alkyl, alkenyl, aryl, cyclyl, heteroaryl, heterocyclyl, alkoxyl, nitro, amino, cyano, or halogen.

Shown in Table 1 are exemplary compounds:

TABLE 1
Benzothiazolium compounds

| Compound | |
|---|---|
| Number | Structure |
| 1 | 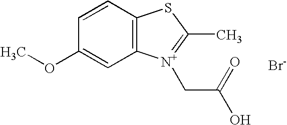 |
| 2 | 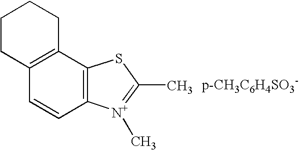 |
| 3 | 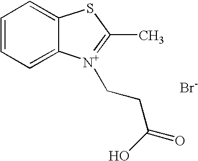 |
| 4 | 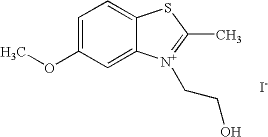 |
| 5 | 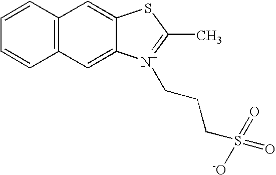 |
| 6 | 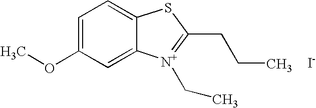 |
| 7 | 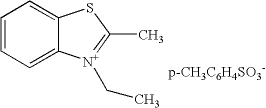 |
| 8 | 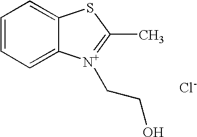 |
| 9 | 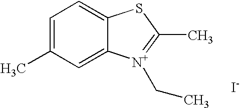 |
| 10 | 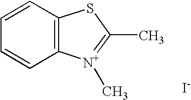 |
| 11 | 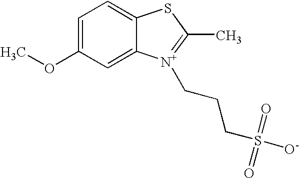 |
| 12 | 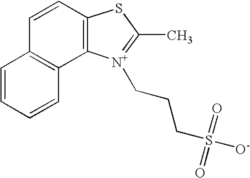 |
| 13 | 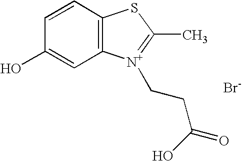 |
| 14 | 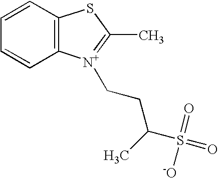 |
| 15 | 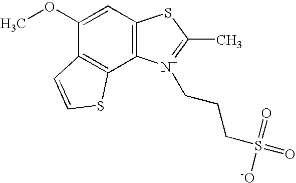 |
| 16 | 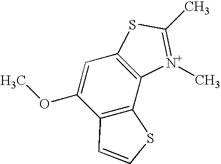 |
| 17 | 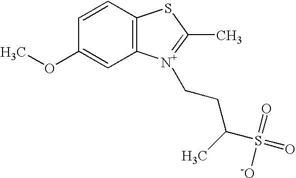 |
| 18 | 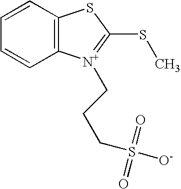 |
| 19 | 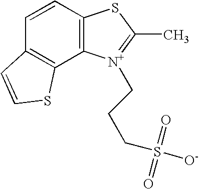 |
| 20 | 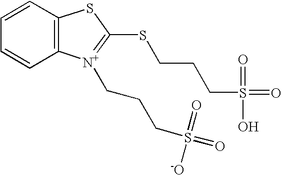 |
| 21 | 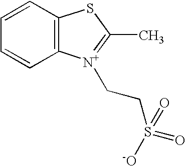 |
| 22 | 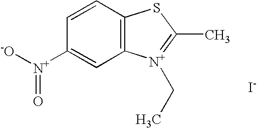 |
| 23 | 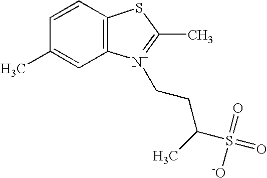 |
| 24 | 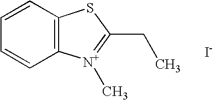 |
| 25 | 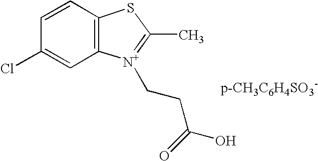 |
| 26 | 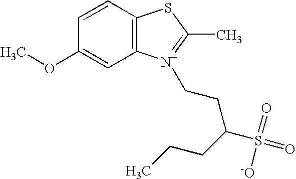 |
| 27 | 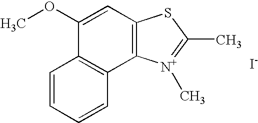 |
| 28 | 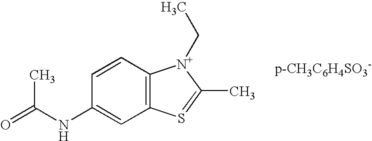 |
| 29 | 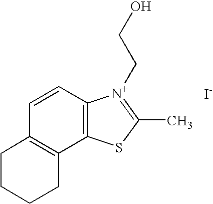 |
| 30 | 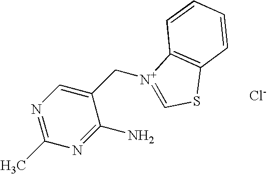 |
| 31 | 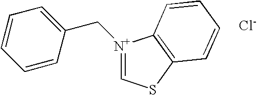 |
| 32 | 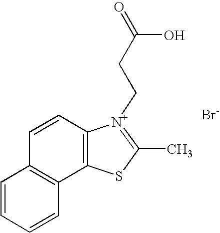 |
| 33 | 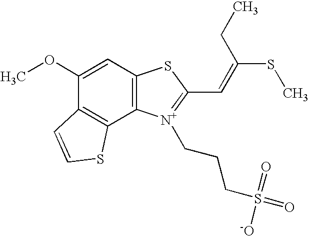 |
| 34 | 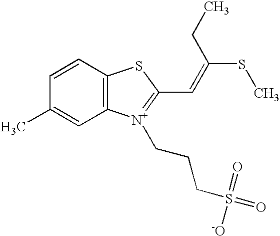 |
| 35 | 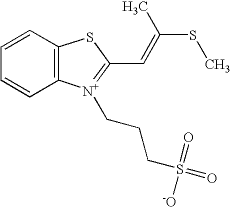 |
| 36 | 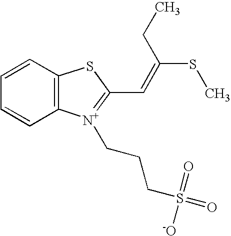 |
| 37 | 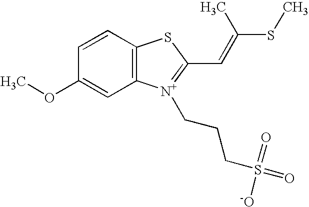 |
| 38 | 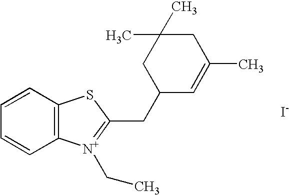 |
| 39 | 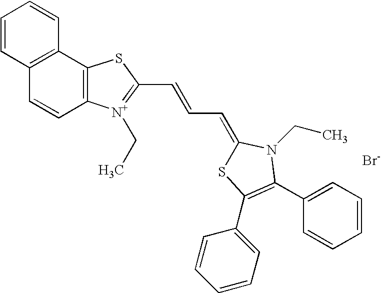 |
| 40 | 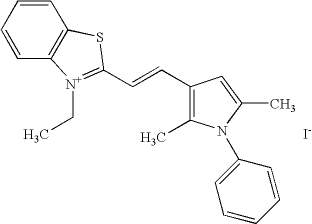 |
| 41 | 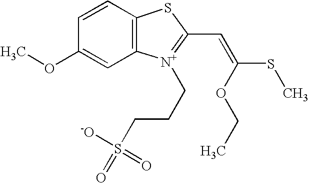 |
| 42 | 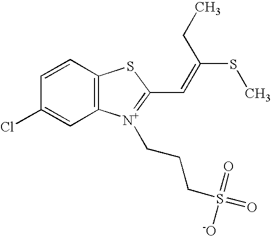 |
| 43 | 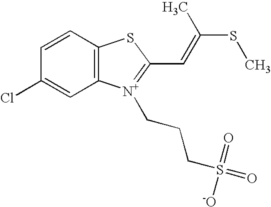 |
| 44 | 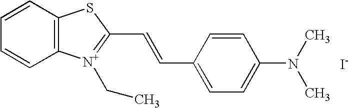 |
| 45 | 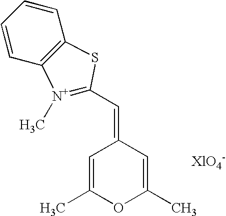 |
| 46 | 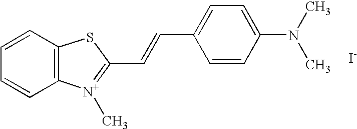 |
| 47 | 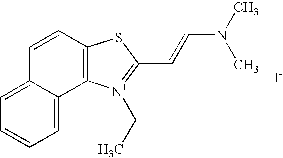 |
| 48 | 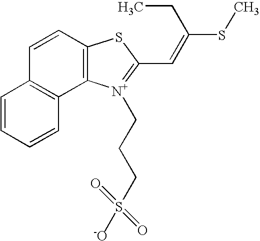 |
| 49 | 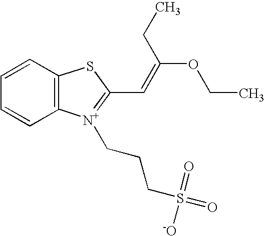 |
| 50 | 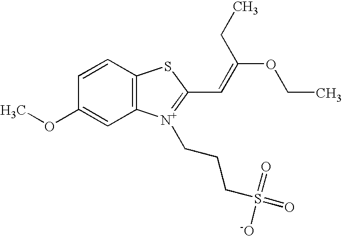 |
| 51 | 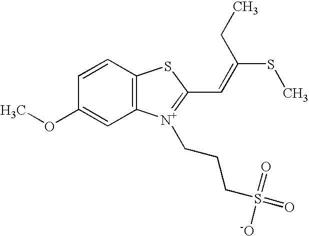 |
| 52 | 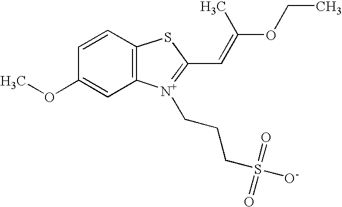 |
| 53 | 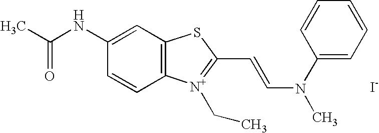 |
| 54 | 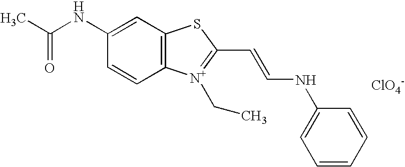 |
| 55 | 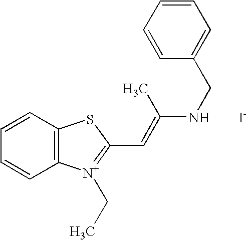 |
| 56 | 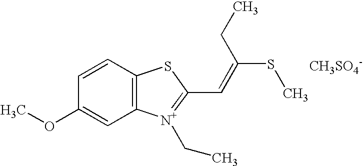 |
| 57 | 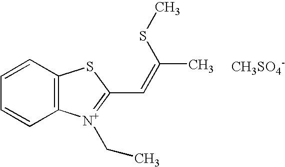 |
| 58 | 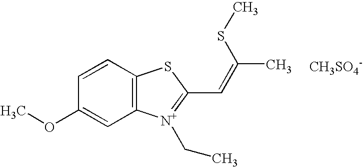 |
| 59 | 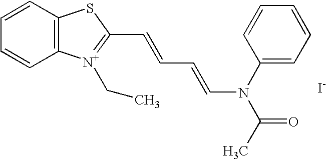 |
| 60 | 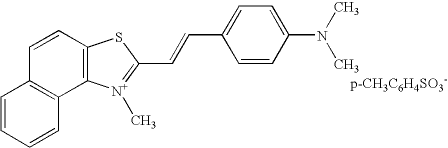 |
| 61 | 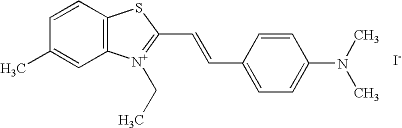 |
| 62 | 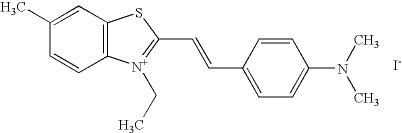 |
| 63 | 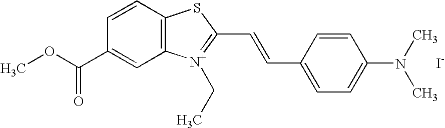 |
| 64 | 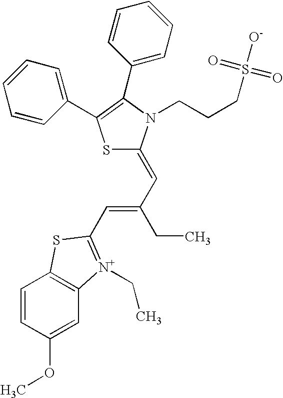 |
| 65 | 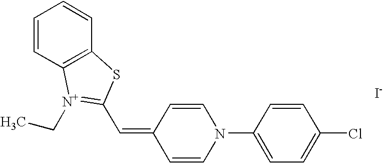 |
| 66 | 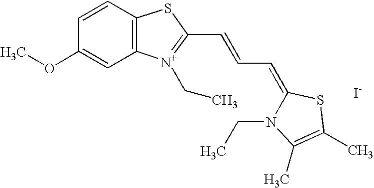 |
| 67 | 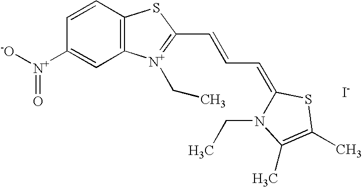 |
| 68 | 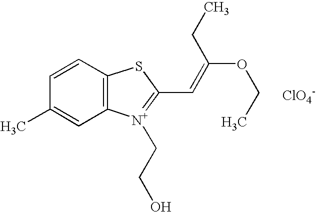 |
| 69 | 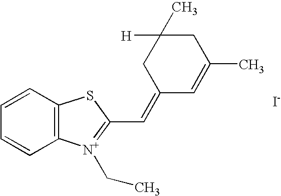 |
| 70 | 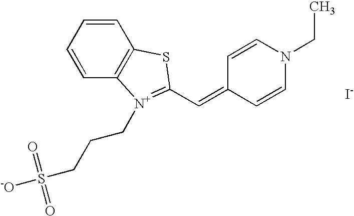 |
| 71 | 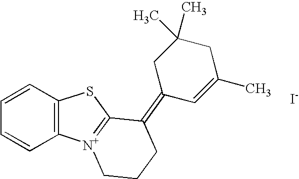 |
| 72 | 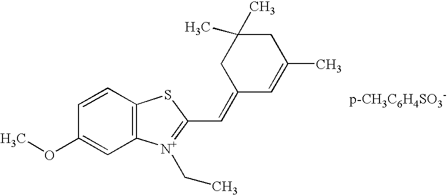 |
| 73 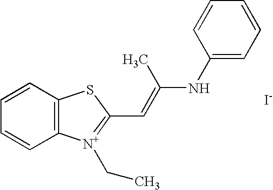 | |
| 74 | 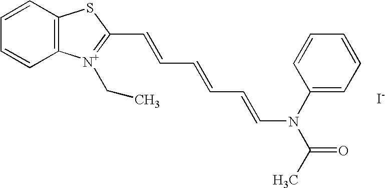 |
| 75 | 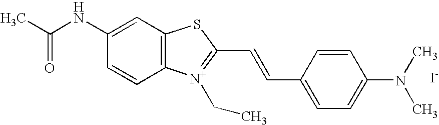 |
| 76 | 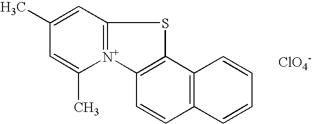 |
| 77 | 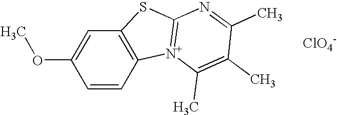 |
| 78 | 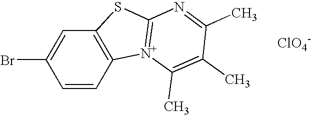 |
| 79 | 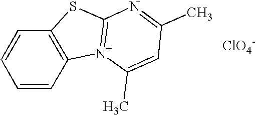 |
| 80 | 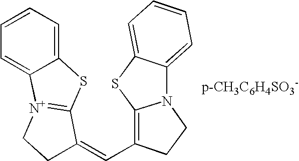 |
| 81 | 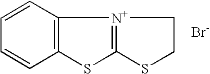 |
| 82 | 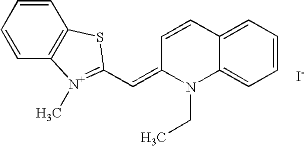 |
| 83 | 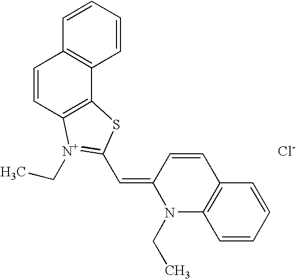 |
| 84 | 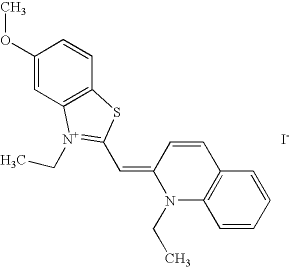 |
| 85 | 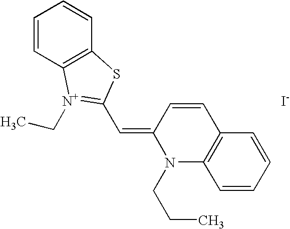 |
| 86 | 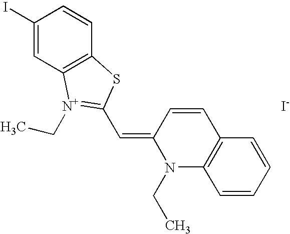 |
| 87 | 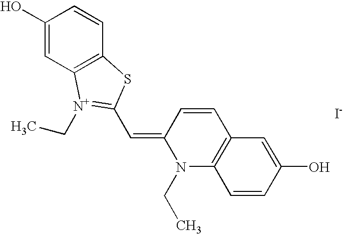 |
| 88 | 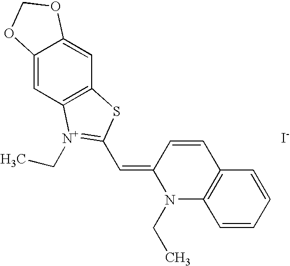 |
| 89 | 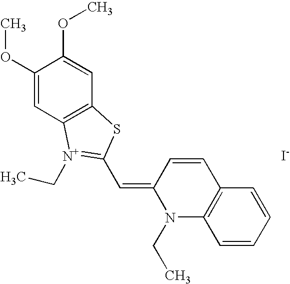 |
| 90 | 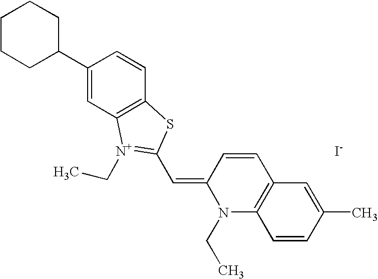 |
| 91 | 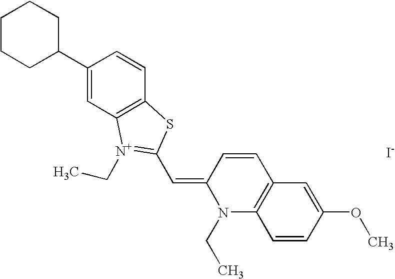 |
| 92 | 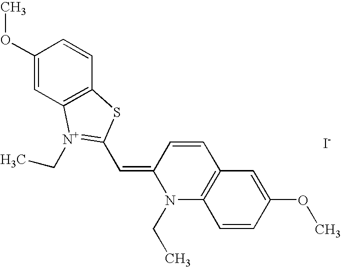 |
| 93 | 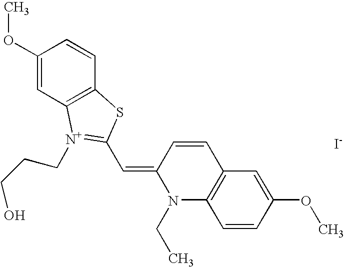 |
| 94 | 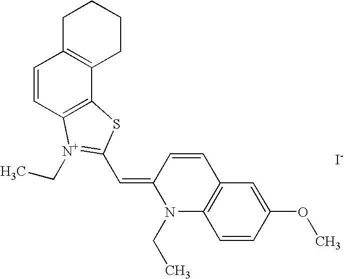 |
| 95 | 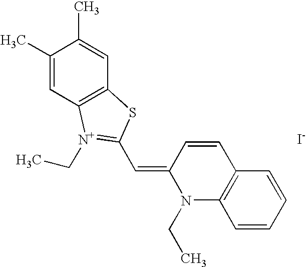 |
| 96 | 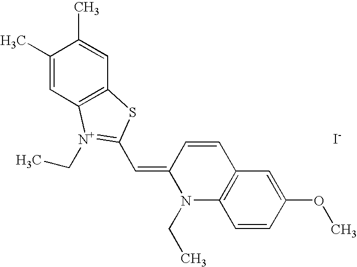 |
| 97 | 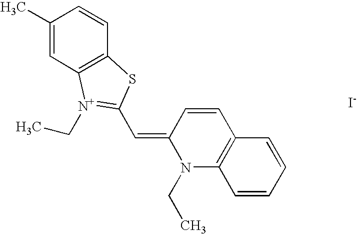 |
| 98 | 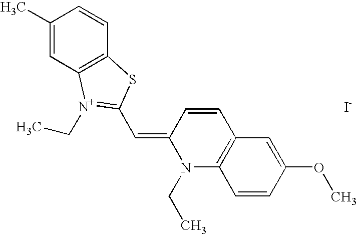 |
| 99 | 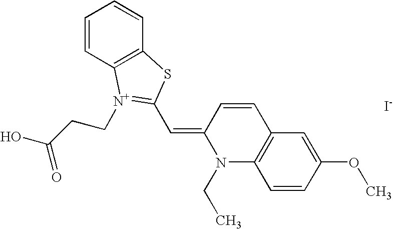 |
| 100 | 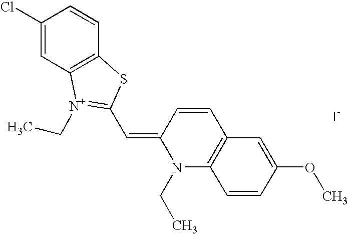 |
| 101 | 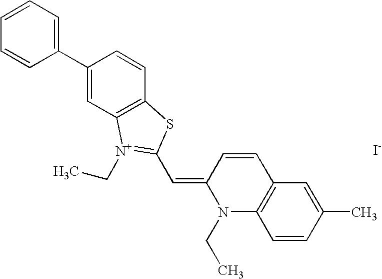 |
| 102 | 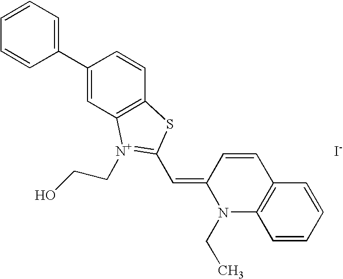 |
| 103 | 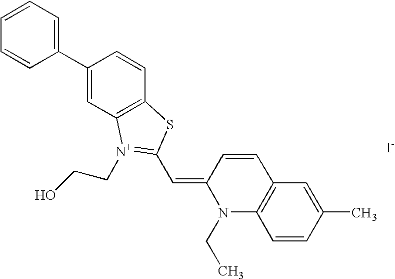 |
| 104 | 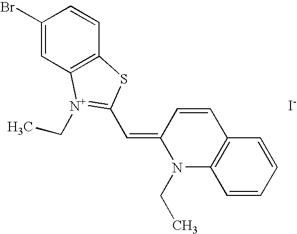 |
| 105 | 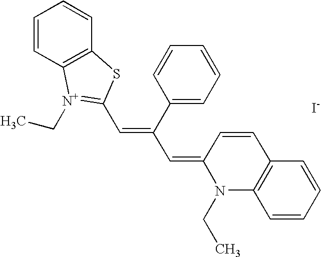 |
| 106 | 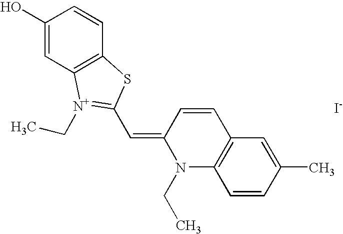 |
| 107 | 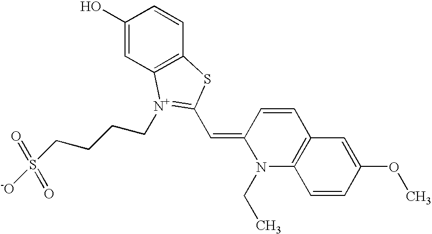 |
| 108 | 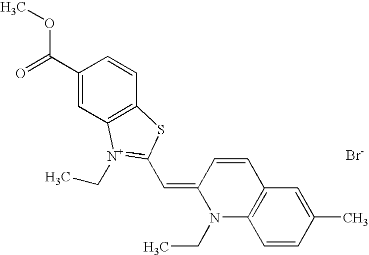 |
| 109 | 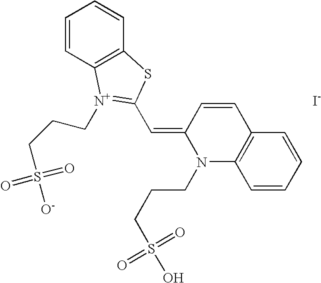 |
| 110 | 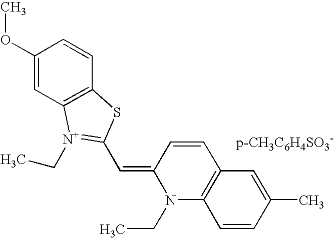 |
| 111 | 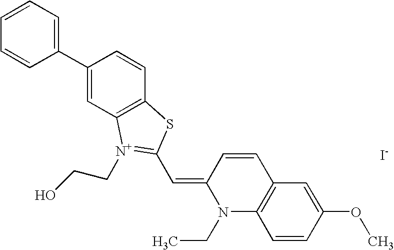 |
| 112 | 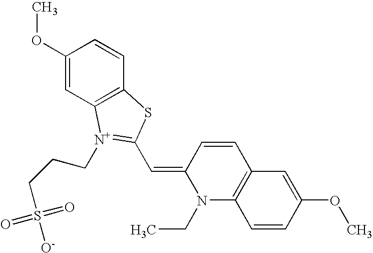 |
| 113 | 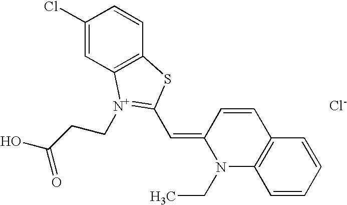 |
| 114 | 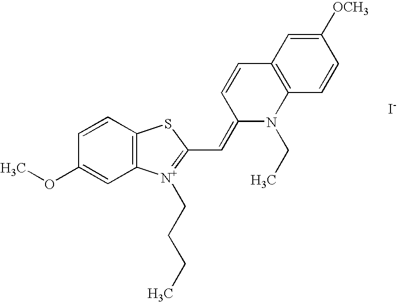 |
| 115 | 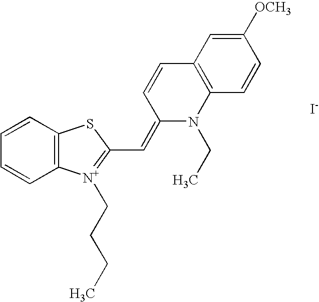 |
| 116 | 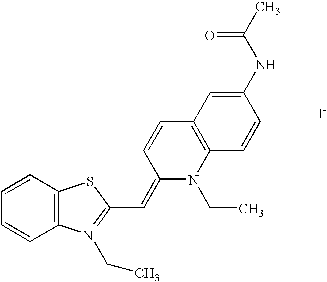 |
| 117 | 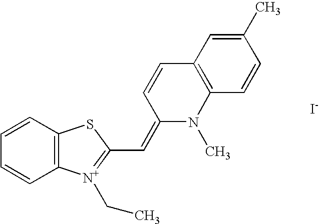 |
| 118 | 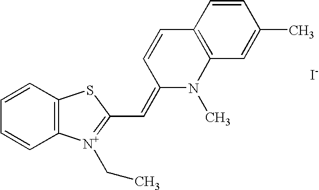 |
| 119 | 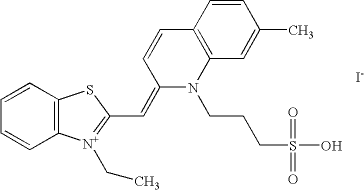 |
| 120 | 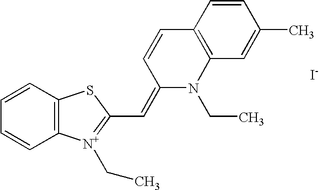 |
| 121 | 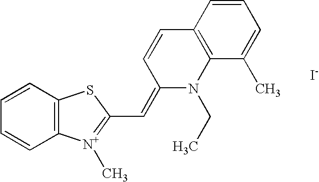 |
| 122 | 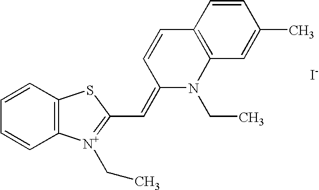 |
| 123 | 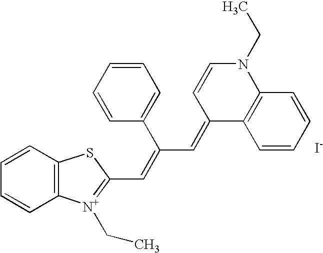 |
| 124 | 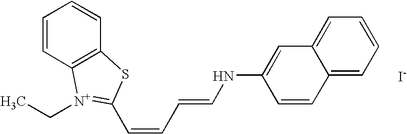 |
| 125 | 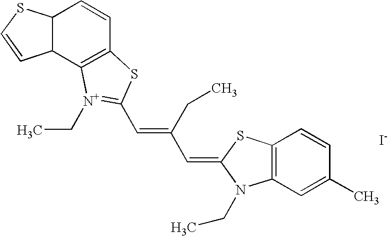 |
| 126 | 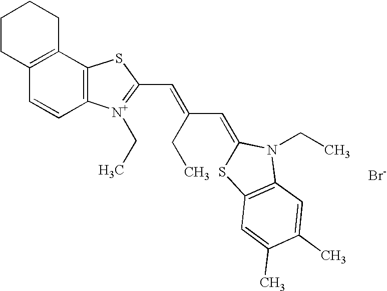 |
| 127 | 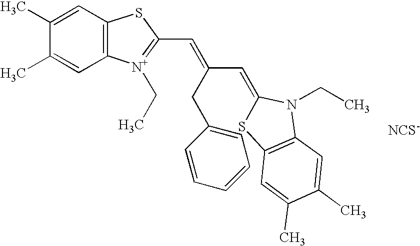 |
| 128 | 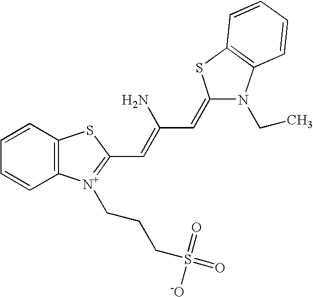 |
| 129 | 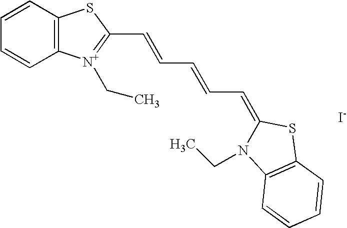 |
| 130 | 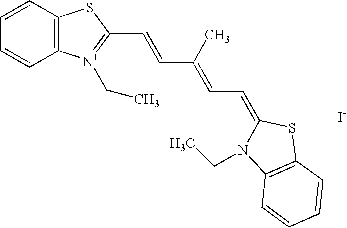 |
| 131 | 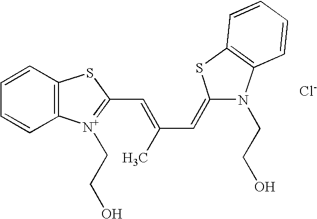 |
| 132 | 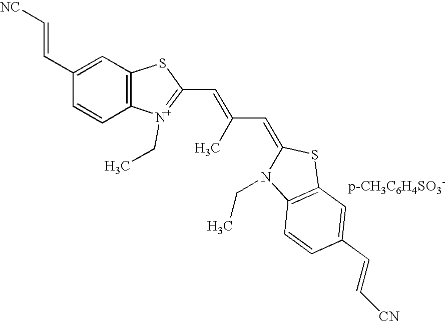 |
| 133 | 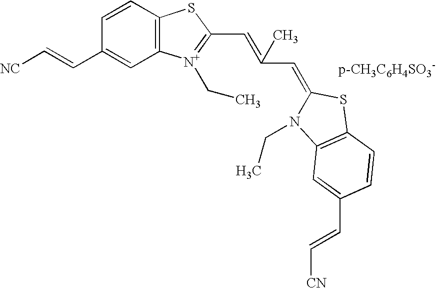 |
| 134 | 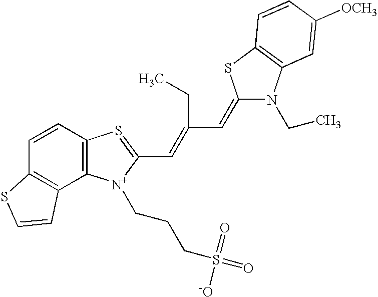 |
| 135 | 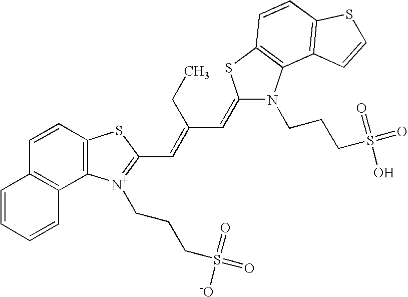 |
| 136 | 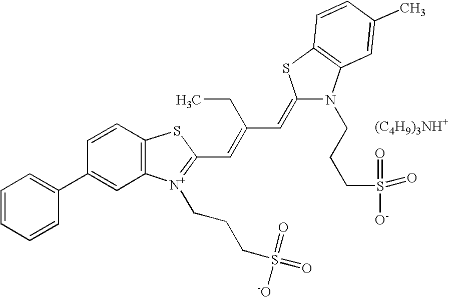 |
| 137 | 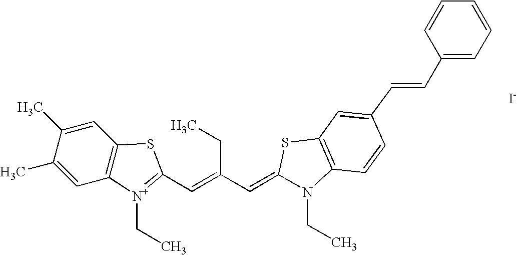 |
| 138 | 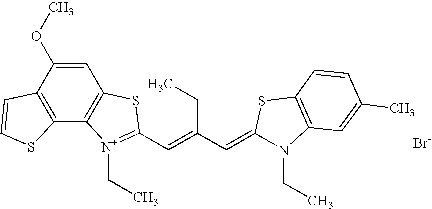 |
| 139 | 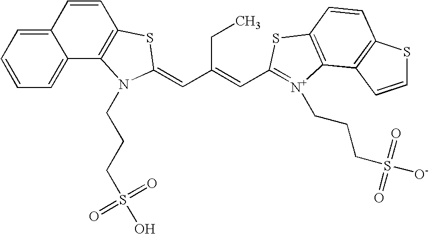 |
| 140 | 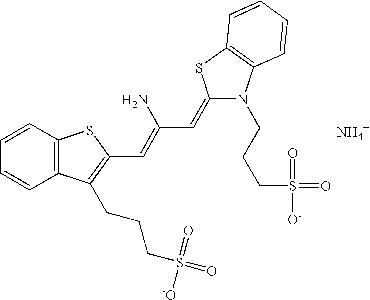 |
| 141 | 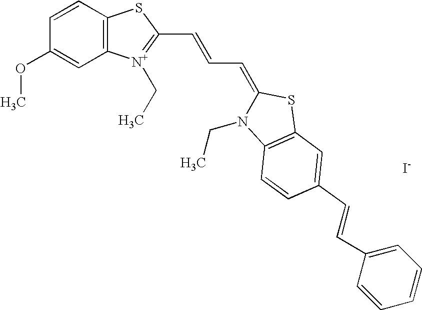 |
| 142 | 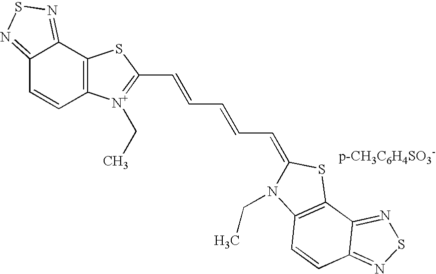 |
| 143 | 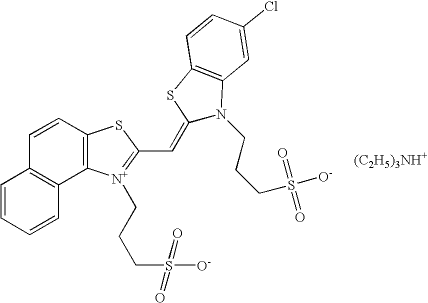 |
| 144 | 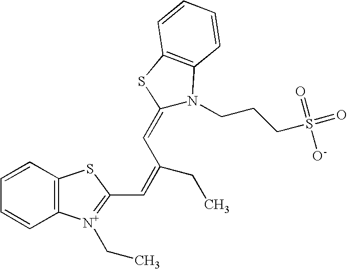 |
| 145 | 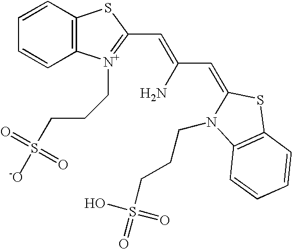 |
| 146 | 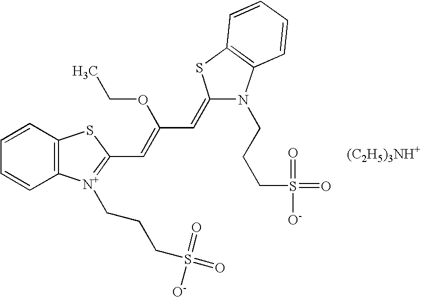 |
| 147 | 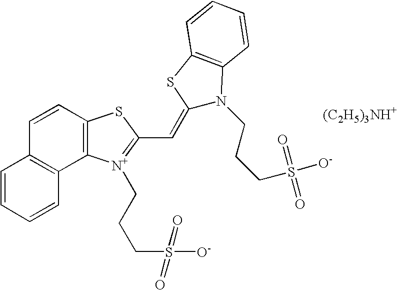 |
| 148 | 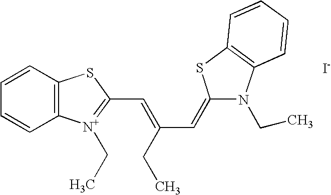 |
| 149 | 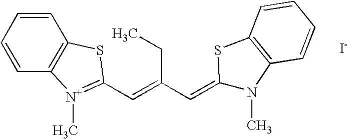 |
| 150 | 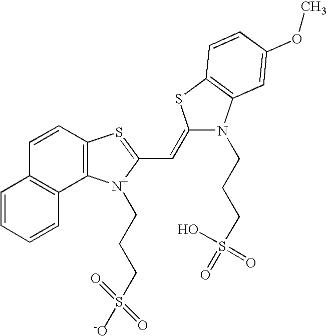 |
| 151 | 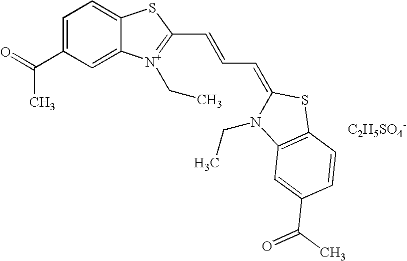 |
| 152 | 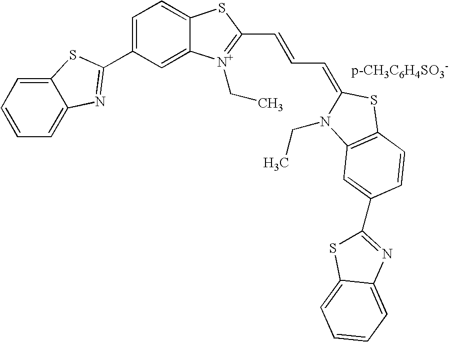 |
| 153 | 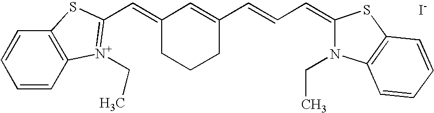 |
| 154 | 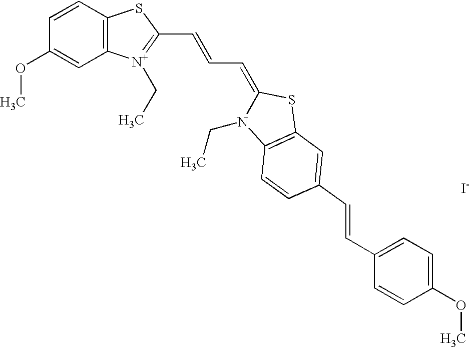 |
| 155 | 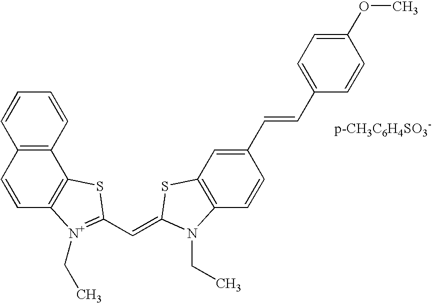 |
| 156 | 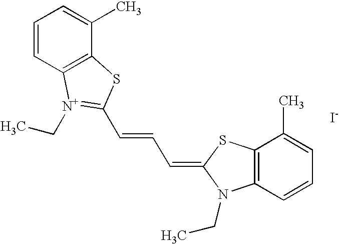 |
| 157 | 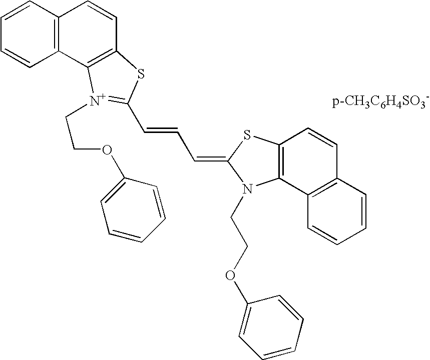 |
| 158 | 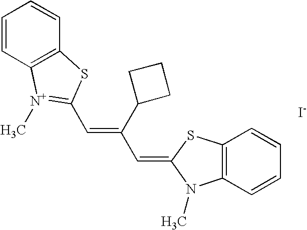 |
| 159 | 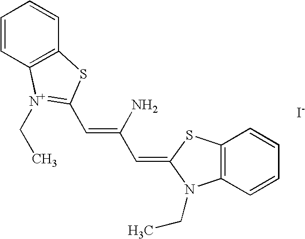 |
| 160 | 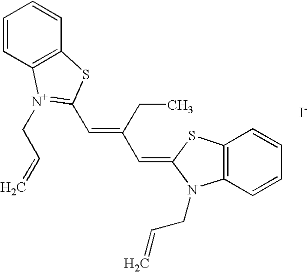 |
| 161 | 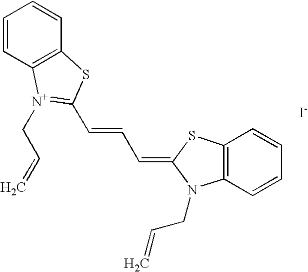 |
| 162 | 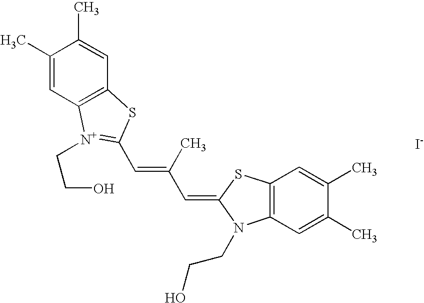 |
| 163 | 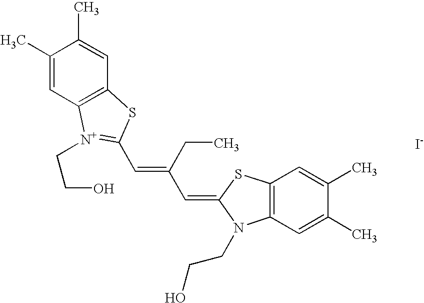 |
| 164 | 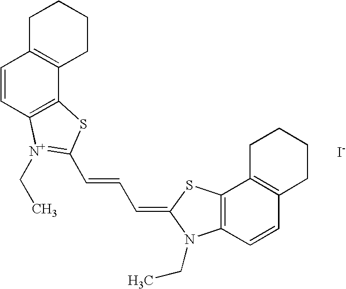 |
| 165 | 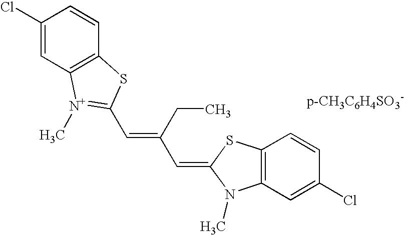 |
| 166 | 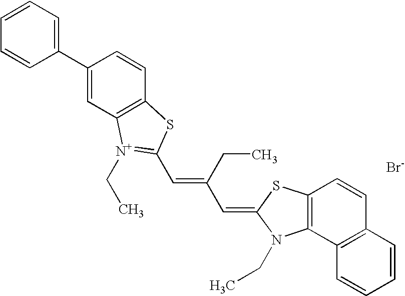 |
| 167 | 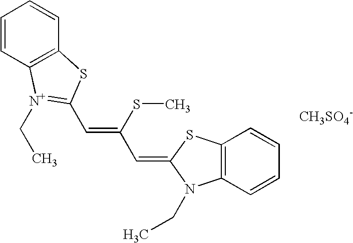 |
| 168 | 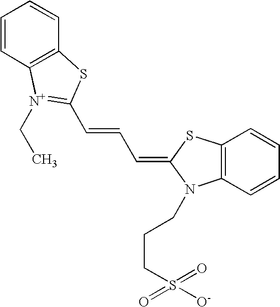 |
| 169 | 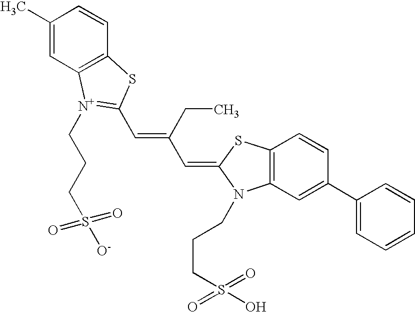 |
| 170 | 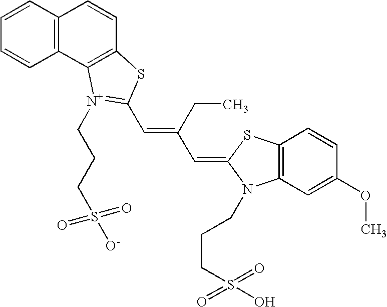 |
| 171 | 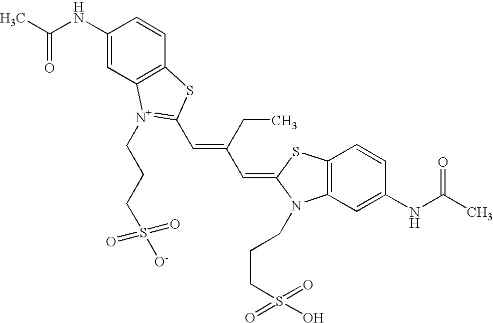 |
| 172 | 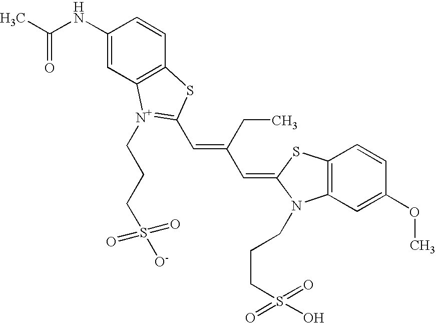 |
| 173 | 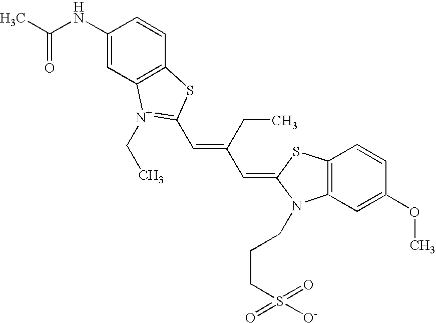 |
| 174 | 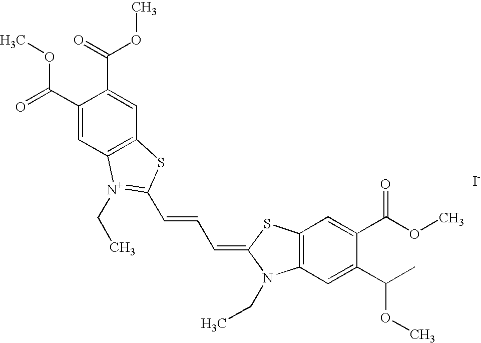 |
| 175 | 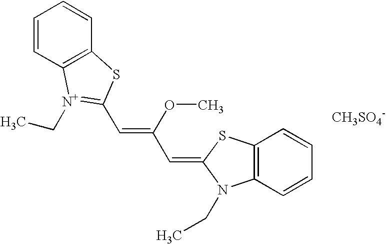 |
| 176 | 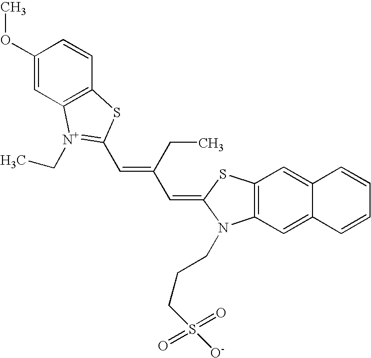 |
| 177 | 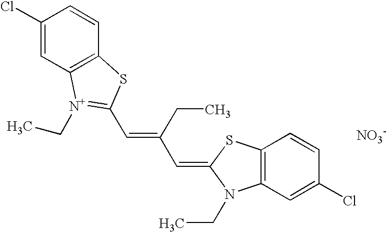 |
| 178 | 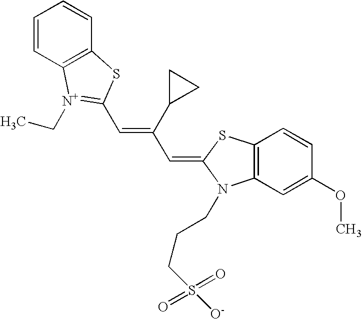 |
| 179 | 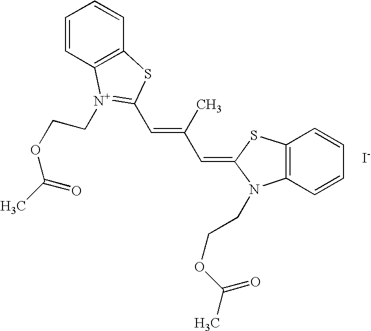 |
| 180 | 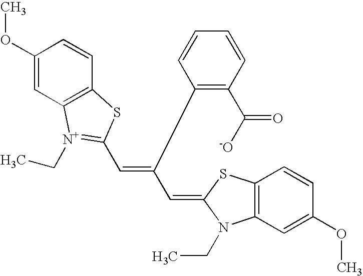 |
| 181 | 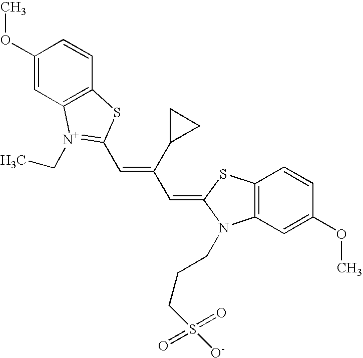 |
| 182 | 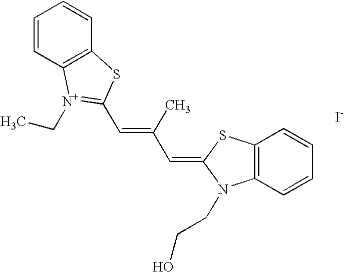 |
| 183 | 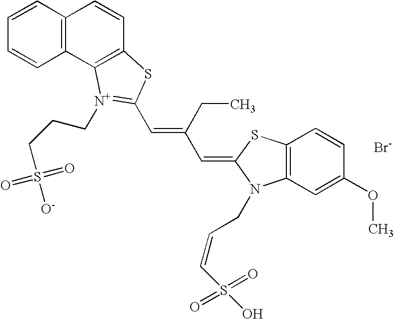 |
| 184 | 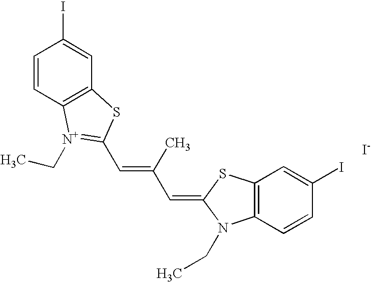 |
| 185 | 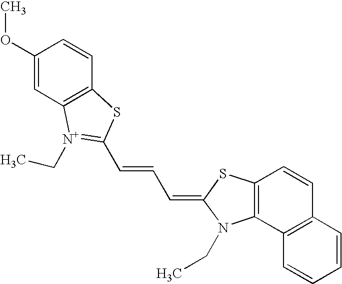 |
| 186 | 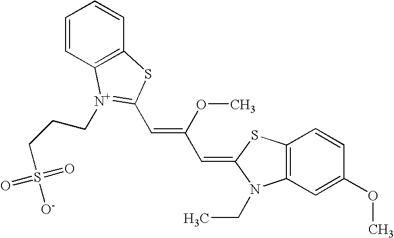 |
| 187 | 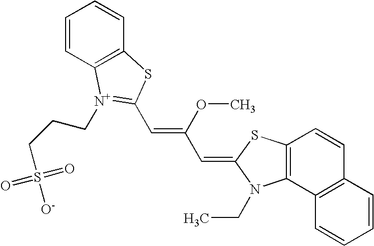 |
| 188 | 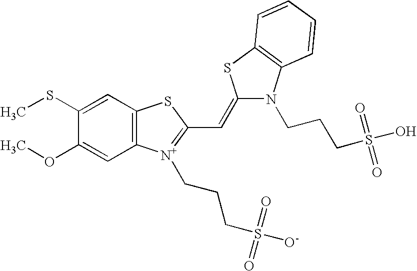 |
| 189 | 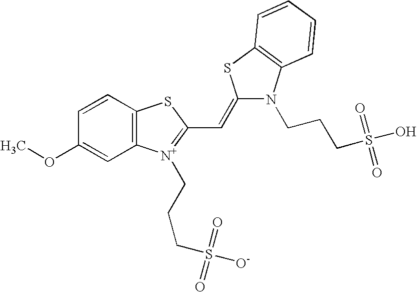 |
| 190 | 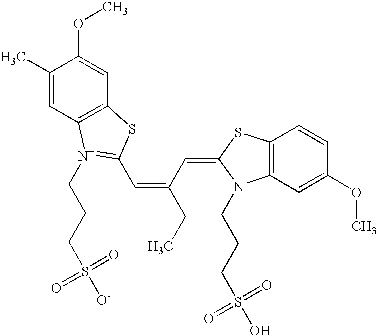 |
| 191 | 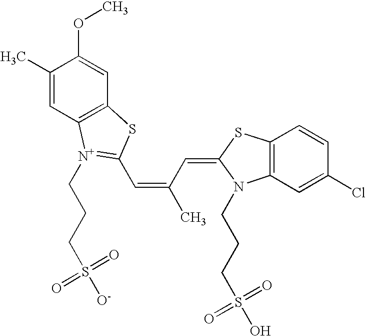 |
| 192 | 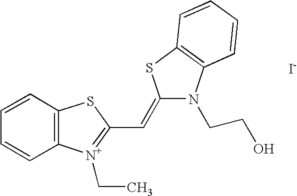 |
| 193 | 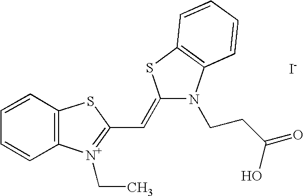 |
| 194 | 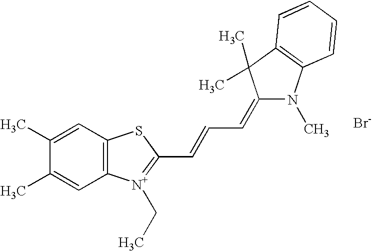 |
| 195 | 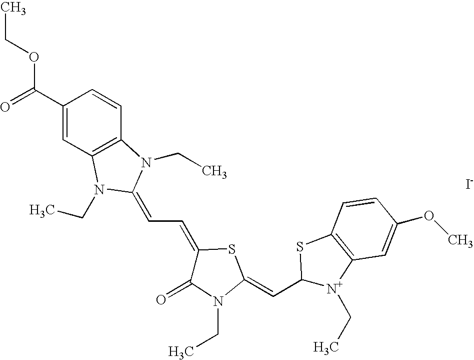 |
| 196 | 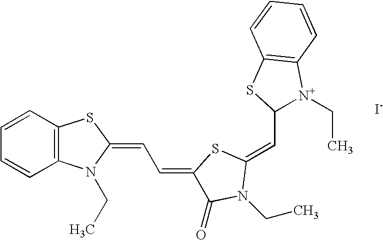 |
| 197 | 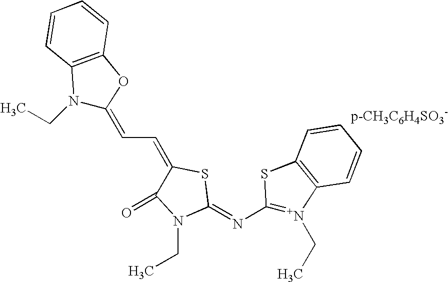 |
| 198 | 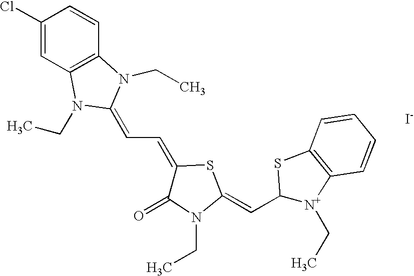 |
| 199 | 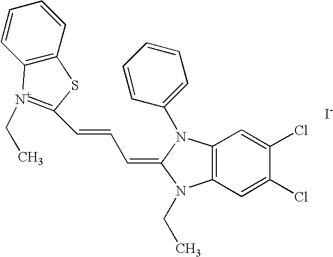 |
| 200 | 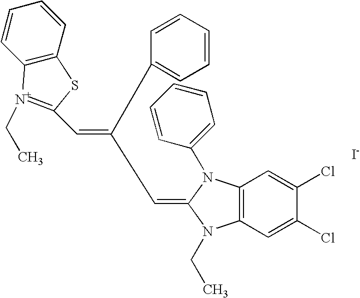 |
| 201 | 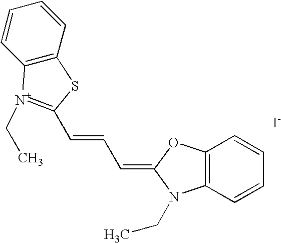 |
| 202 | 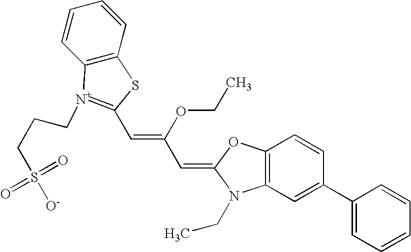 |
| 204 | 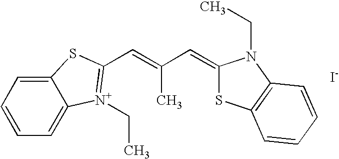 |
| 205 | 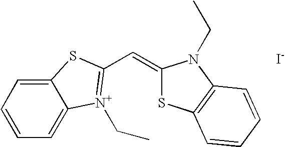 |
| 206 | 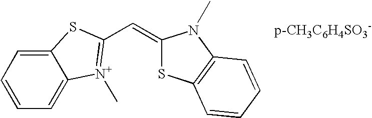 |

The term “alkyl” refers to a straight or branched hydrocarbon, containing 1-10 carbon atoms. Examples of alkyl groups include, but are not limited to, methyl, ethyl, n-propyl, i-propyl, n-butyl, i-butyl, and t-butyl. The term “alkenyl” referes to a straight or branched hydrocarbon, containing 1-10 carbon atoms and one or more double bonds. The term “alkynyl” referes to a straight or branched hydrocarbon, containing 1-10 carbon atoms and one or more triple bonds. The term “alkoxy” refers to an —O-alkyl. The term “alkoxyalkyl” refers to an alkyl group substituted with one or more alkoxy groups. The term “haloalkyl” refers to an alkyl group substituted with one or more halo groups. The term “hydroxyalkyl” refers to an alkyl group substituted with one or more hydroxy groups.

The term “aryl” refers to a 6-carbon monocyclic, 10-carbon bicyclic, 14-carbon tricyclic aromatic ring system wherein each ring may have 1 to 4 substituents. Examples of aryl groups include, but are not limited to, phenyl, naphthyl, and anthracenyl. The term “aryloxy” refers to an —O-aryl. The term “aralkyl” refers to an alkyl group substituted with an aryl group.

The term “cyclyl” refers to a saturated and partially unsaturated cyclic hydrocarbon group having 3 to 12 carbons. Examples of cyclyl groups include, but are not limited to, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having one or more heteroatoms (such as O, N, or S). Examples of heteroaryl groups include pyridyl, furyl, imidazolyl, benzimidazolyl, pyrimidinyl, thienyl, quinolinyl, indolyl, and thiazolyl. The term “heteroaralkyl” refers to an alkyl group substituted with a heteroaryl group.

The term “heterocyclyl” refers to a nonaromatic 5-8 membered monocyclic, 8-12 membered bicyclic, or 11-14 membered tricyclic ring system having one or more heteroatoms (such as O, N, or S). Examples of heterocyclyl groups include, but are not limited to, piperazinyl, pyrrolidinyl, dioxanyl, morpholinyl, and tetrahydrofuranyl.

The term “negatively charged group” refers to a functional group having one or more negative charges. Examples of negatively charged groups include, but are not limited to, —CO₂⁻ or —SO₃⁻, and PO₃²⁻.

Alkyl, alkenyl, alkynyl, cyclyl, heterocyclyl, aryl, heteroaryl, aralkyl, heteroaralkyl, alkoxy, and aryloxy mentioned herein include both substituted and unsubstituted moieties. Examples of substituents include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkyl, alkenyl, alkynyl, alkyloxy, aryl, heteroaryl, cyclyl, heterocyclyl, in which alkyl, alkenyl, alkynyl, alkyloxy, aryl, heteroaryl, cyclyl, and heterocyclyl are optionally further substituted with alkyl, aryl, heteroaryl, halogen, hydroxyl, amino, mercapto, cyano, or nitro.

The compounds described above include their pharmaceutically acceptable salts and prodrugs, if applicable. Such a salt can be formed between a negatively charged ionic group in a benzothiazolium compound (e.g., carboxylate) and a positively charged counterion (e.g., sodium, potassium, calcium, or magnesium). Likewise, a positively charged ionic group in a benzothiazolium compound (e.g., ammonium) can also form a salt with a negatively charged counterion (e.g., chloride, bromide, or iodide). Examples of prodrugs include esters and other pharmaceutically acceptable compounds, which, upon administration to a subject, are each capable of providing one of the benzothiazolium compounds described above.

Another aspect of this invention relates to a method of inhibiting production of nitric oxide, by administering to a subject in need thereof an effective amount of one of the compounds described above.

Still another aspect of this invention relates to a method of treating coronaviral infection by administering to a subject in need thereof an effective amount of one of the compounds described above.

Also within the scope of this invention is a composition containing one or more of the benzothiazolium compounds described above for use in treating coronaviral infection, as well as the use of such a composition for the manufacture of a medicament for treating coronaviral infection.

The details of many embodiments of the invention are set forth in the description below. Other features, objects, and advantages of the invention will be apparent from the description and the claims.

### DETAILED DESCRIPTION

The above-described benzothiazolium compounds are commercially available. They can also be prepared by methods well known in the art. For example, benzothiazole is first substituted with an alkyl, alkenyl, or aryl group by a conventional reaction, such as a substitution reaction, or a coupling reaction. The substituted benzothiazole compound reacts with an alkylating agent, e.g., methyl iodide, or methyl-p-toluenesulfonate, to afford an alkylated benzothiazolium compound.

The methods described above may also additionally include steps, either before or after the steps described specifically herein, to add or remove suitable protecting groups in order to ultimately allow synthesis of the benzothiazolium compounds. In addition, various synthetic steps may be performed in an alternate sequence to give the desired compounds. Synthetic chemistry transformations and protecting group methodologies (protection and deprotection) useful in synthesizing applicable benzothiazolium compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

A benzothiazolium compound thus obtained can be further purified by column chromatography, high performance liquid chromatography, or crystallization.

The above described benzothiazolium compounds may contain a non-aromatic double bond and one or more asymmetric centers. Thus, they can occur as racemic mixtures, single enantiomers, individual diastereomers, diastereomeric mixtures, and cis- or trans-isomeric forms. All such isomeric forms are contemplated.

The benzothiazolium compounds suppressing NO production. Thus, this invention includes methods of suppressing NO production by administering to a subject in need thereof an effective amount of one of the above-described compounds. The term “an effective amount” refers to the amount of the compound which is required to confer one of the above-described effects in the subject. Effective amounts may vary, as recognized by those skilled in the art, depending on route of administration, excipient usage, and the possibility of co-usage with other agents.

Also within the scope of this invention is a method of inhibiting expression of iNOS or lowering production of TNF a and IL-6. The method includes administering to a subject in need of inhibiting NO production an effective amount of one of the benzothiazolium compounds described above.

This invention also covers a method for treating coronavirus infection. The method includes administering to a subject in need thereof an effective amount of one of the benzothiazolium compounds described above and a pharmaceutically acceptable carrier. The term “coronavirus” is well known in the art. It refers to a genus of pleomorphic viruses that resemble coronas when viewed with a microscope. Examples of coronavirus include, but are not limited to, human CoV 229E, transmissible gastroenteritis virus (TGEV), mouse hepatitis virus, bovine CoV, infectious bronchitis virus, and severe acute respiratory syndrome virus. The term “treating” refers to administering the extract to a subject that is infected with coronavirus, or has a symptom of the infection, or has a predisposition toward the infection, with the purpose to cure, heal, alleviate, relieve, alter, remedy, ameliorate, improve, or affect the infection, the symptoms of the infection, or the predisposition toward the infection.

To practice the method of the present invention, a composition having one of the benzothiazolium compounds describe above can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol and water. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having an active benzothiazolium compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active benzothiazolium compound. Examples of other carriers include colloidal silicon oxide, magnesium stearate, cellulose, sodium lauryl sulfate, and D&C Yellow # 10.

The benzothiazolium compounds describe above can be preliminarily screened by an in vitro assay for their activity, e.g., inhibiting NO production. Compounds that demonstrate high activity in the preliminary screening can further be screened for their efficacy in treating coronaviral infection by in vivo assays. For example, a test compound can be administered to an animal (e.g., a mouse model) affected with coronavirus and its therapeutic effects are then accessed. Based on the results, an appropriate dosage range and administration route can also be determined.

The specific examples below are to be construed as merely illustrative, and not limitative of the remainder of the disclosure in any way whatsoever. Without further elaboration, it is believed that one skilled in the art can, based on the description herein, utilize the present invention to its fullest extent.

Preparation of Benzothiazolium Compounds

Compounds 1-204 were purchased from Chemical Diversity Labs, Inc. (San Diego). Compound 205 was purchased from Sigma Aldrich Co. (U.S.A.). Compound 206 was synthesized as follows:

### Synthesis of 3-methyl-2-(3-methyl-3H-benzothiazol-2-ylidenemethyl)-benzothiazol-3-ium p-toluenesulfonate (Compound 206)

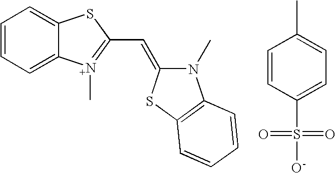

A mixture of 2,2-methylenebisbenzothiazole (0.565 g, 2 mmol) and methyl p-toluenesulfonate (0.466 g, 2.5 mmol) was heated by microwave (160° C., 5 min) to give a gum. The gum was washed with acetone repeatedly to yield the desired compound as a yellow crystal (0.65 g, 67%).

Biological Assays

RAW 264.7 macrophage cells were maintained in sodium pyruvate-free Dubelcco's modified Eagle medium (Hyclone) with 4 mM glutamine containing 1% non-essential amino acids (Biological Industries, Israel) and 10% heat-inactivated fetal bovine serum (Hyclone) or 10% fetal calf serum (Biological Industries, Israel) in culture plates. The cells were scraped off the culture plates for passage without any trypsin or EDTA treatment, and grown in an incubator at 37° C. and 5% CO₂. FuGene6™ was obtained from Roche (German) and lipopolysaccharide of E. coli O111:B4 from Chemicon International (California, U.S.A.).

A murine iNOS promoter-Luc, a human iNOS promoter-Luc (pGL3-8296), and a murine cyclooxygenase II promoter-Luc plasmid were generously provided by Drs. Charles J. Lowenstein (John Hopkines University), Joel Moss (National Institute of Health) and Yu-Chih Liang (Taipei Medical University), respectively. CMX-β-gal plasmid containing the E. coli β-galactosidase coding sequence was used for transfection efficiency control.

Inhibition of iNOS Promoter Activity

RAW 264.7 cells (or A549 cells) were seeded in 24-well plates (9×10⁴ cells/well). The cells reached to 90-95% confluence in the above-described medium with antibiotics within 24 h and transfected with murine or human iNOS promoter-luciferase reporter plasmids (100 ng/well) using FuGene6 (Roche, Co.) following the protocol provided by the manufacturer. Transfection efficiencies were normalized by co-transfection with 100 ng/well of the β-galactosidase expression plasmid. After 24 h incubation, the medium was replaced with the above-described medium containing LPS (5 μg/ml)/IFNγ (20 ng/ml) and test compounds were added at 10 μM. After 24 h incubation, the medium was removed, cell lysis buffer was added (50 μl/well) and the lysates were subjected to the luciferase assay according to the manufacturer's instructions (Tropix).

The luciferase assay was performed using a luciferase assay system according to the manufacturer's instructions. Luminescence was measured in a TopCount.NXT™ Microplate Scintillation and Luminescence Counter (Packard, Inc.). The results were normalized to β-galactosidase activity derived from co-transfected LacZ gene under the control of a constitutive promoter.

The results show that all test compounds inhibited iNOS promoter activity, thereby suppressing iNOS expression.

Inhibition of Nitric Oxide Production

RAW 264.7 cells were seeded (70,000 cells/well) and cultured in 96-well culture plate. After 24 h incubation, the medium was replaced with a medium containing stimuli of LPS (5 ug/ml)/IFNγ (20 ng/ml) and the test compounds were added at different concentrations. After 18-24 h, the supernatants were subjected to the measurement of nitric oxide production using the Nitrate/Nitrite assay kit (Cayman Chemical). Nitric oxide levels were measured as the accumulation of nitrite and nitrate in the incubation medium. Nitrate was reduced to nitrite with nitrate reductase and determined spectrophotometrically with Griess reagent at OD₄₀₅. The attached cells were subjected to cytotoxicity measurement using a MTS assay. The results show that a number of benzothiazolium compounds effectively inhibited production of NO.

Cytokine Measurement

TNF α and IL-6 proteins were measured in cell culture supernatants using an ELISA kit from R & D Systems Inc. (U.S.A.) according to the manufacturer's instructions. The results show that several test compounds effectively inhibited production of TNF α and production of IL-6.

Inhibition of iNOS and Cyclooxygenase II Expression

Levels of iNOS, cyclooxygenase II, and β-actin (control) were measured by immunoblotting with anti-iNOS antibody (Biomol), anti-cyclooxygenase II antibody (Upstate), and anti-β-actin antibody (Chemicon), respectively. The cell lysates were subjected to SDS-PAGE and the separated proteins were electrophoretically transferred to nitrocellulose membranes. The membranes were incubated, respectively, with blocking solution for 1 h, primary antibody for 2 h, and secondary antibody for 1 h, and wash procedures were carried out. Antigen-antibody complexes were detected using ECL detection reagents (Perkin Elmer, Western Blot Chemiluminescence Reagent Plus) according to the manufacturer's instructions. The results show that several benzothazolium compounds lowered both iNOS and cyclooxygenase II levels.

Anti-SARS CoV Assay

Fluorogenic peptide substrate Dabcyl-KTSAVLQSGFRKME-Edans was obtained from Biogenesis (Taiwan). Expression and purification of SARS CoV main protease were performed as described in Kuo, et al. Biochemical and Biophysical Research Communications, 2004, 318: 862-867.

A mixture containing 50 nM SARS protease, 6 μM fluorogenic peptide substrate in a buffer of 12 mM Tris-HCl (pH 7.5), 120 mM NaCl, 0.1 mM EDTA, and 1 mM DTT plus 7.5 mM b-ME was prepared. From this mixture, a series of solutions having different concentrations of a test compound (ranging from 0 to 50 μM) were obtained. The fluorescence change of the solutions was measured using a 96-well fluorescence plate reader. The IC₅₀ values were calculated using the following equation:
A(I)=A(0)×{1−[I/(I+IC₅₀)]}
where A(I) is the enzyme activity at compound concentration I; A(O) is the enzyme activity in the absence of the compound; and I is the compound concentration.

A number of compounds inhibited the activity of SARS coronavirus. Unexpectedly, some of the compounds have IC₅₀ values lower than 50 μM.

Anti-Human CoV 229E Assay

Human fibroblast MRC-5 cells were seeded to 96-well plates (70,000 cells/well) and incubated at 37° C. for 48 hours. The culture medium was replaced with a medium containing 229E virus. After 1 hour, a test compound was added to the plate and incubated at 37° C. for 64 hours. After washed with PBS three times, the cells were fixed and stained with 0.1% crystal violet and analyzed in an automatic microtiter plate reader at OD₅₇₀ to measure relative cell numbers with respect to control, in which no test compound was added. The results show that several benzothiazolium compounds effectively inhibited human CoV 229E.

Anti-TGEV Assay

ST cells were seeded into a 96-well plate (50,000 cells/well) and incubated at 37° C. overnight. The culture medium was replaced with 2% fetal bovine serum containing a test compound (final concentration of 10 μM) and incubated at 37° C. for 2 hrs before TGEV infection (>5 m.o.i.). The culture plates were subjected to indirect fluorescent antibody (IFA) 7 hours after infection, washed with PBS three times, fixed by 80% acetone, and then stored at −20° C. The cells were subjected to IFA staining using antibody against TGEV S and N proteins as the primary antibody and anti-mouse Ig conjugate FITC as the secondary antibody. Fluorescence intensities were measured on a Victor II plate reader. The results show that a number of benzothiazolium compounds effectively inhibited TGEV.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to above-described compounds also can be made, screened for the above-described activities and used to practice this invention. Thus, other embodiments are also within the claims.

What is claimed is:

1. A method of inhibiting production of nitric oxide, comprising administering to a subject in need thereof an effective amount of a compound of the following formula: 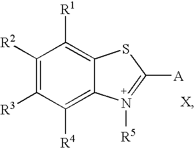
wherein
each of R¹, R², R³, and R⁴, independently, is H, halo, alkyl, alkenyl, akynyl, alkoxyl, aryl, heteroaryl, cylcyl, heterocyclyl, nitro, cyano, amino, amido, or alkoxycarbonyl, or R¹, R², and the two carbon atoms to which they are attached, together form a 3-8 membered ring containing 0, 1, 2, or 3 heteroatoms, or R², R³, and the two carbon atoms to which they are attached, together form a 3-8 membered ring containing 0, 1, 2, or 3 heteroatoms, or R³, R⁴, and the two carbon atoms to which they are attached, together form a 3-8 membered ring containing 0, 1, 2, or 3 heteroatoms;
R⁵ is H, alkyl, aryl, heteroaryl, cyclyl, or heterocyclyl;
A is H, alkyl, aryl, heteroaryl, cyclyl, heterocyclyl, alkoxy, amino, halo, mercapto, thioalkoxy, 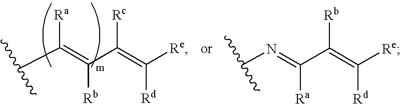
in which m is 0, 1, 2, or 3; each of R^{a}, R^{b}, R^{c}, R^{d}, and R^{e} independently is H, alkyl, akenyl, akynyl, halo, aryl, heteroaryl, cyclyl, heterocylyl, alkoxy, mercapto, thioalkoxy, amino, amido, or R^{d}, R^{e}, and the carbon atom to which they are attached, together form a 3-8 membered ring containing 0, 1, 2, or 3 heteroatoms, which is optionally fused with aryl, heteroaryl, cyclyl, or heterocyclyl;
optionally A, R⁵, the N atom to which R⁵ is attached, and the carbon atom to which A is attached, together form a 4-8 membered ring containing 1, 2, or 3 heteroatoms; and
X is an anion or a negatively charged group attached to an atom of R¹, R², R³, R⁴, R⁵, or A.

2. The method of claim 1, wherein X is a negatively charged group attached to an atom of R¹, R², R³, R⁴, R⁵, or A, the negatively charged group being selected from —CO₂, —SO₃—, or —PO₃²⁻.

3. The method of claim 1, wherein X is I⁻, Br⁻, Cl⁻, ClO₄—, C₂H₅SO₄⁻, or

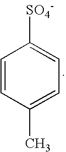

4. The method of claim 1, wherein A is H or alkyl.

5. The method of claim 4, wherein R⁵ is alkyl.

6. The method of claim 1, wherein R⁵ is alkyl.

7. The method of claim 1, wherein A is

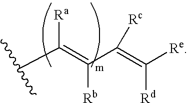

8. The method of claim 7, wherein one of R^{d} and R^{e} is substituted or unsubstituted phenyl.

9. The method of claim 7, wherein one of R^{d} and R^{e} is thioalkyl, amino, or amido.

10. The method of claim 7, wherein R^{d}, R^{e}, and the carbon atom to which they are attached, together form a 3-8 membered ring containing 0, 1, or 2 heteroatoms.

11. The method of claim 10, wherein A is 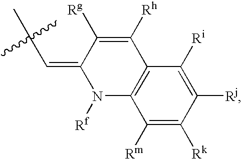
R^{f} being H, alkyl, alkenyl, aryl, cyclyl, heteroaryl, or heterocyclyl; and each of R^{g}, R^{h}, Rⁱ, R^{j}, R^{k}, and R^{m}, independently being H, alkyl, alkenyl, aryl, cyclyl, heteroaryl, heterocyclyl, alkoxyl, halogen, nitro, or cyano.

12. The method of claim 11, wherein each of R^{f} and R⁵ is alkyl.

13. The method of claim 10, wherein A is 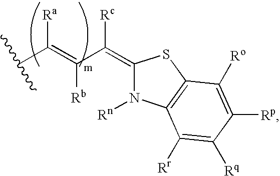
Rⁿ being H, alkyl, alkenyl, aryl, cyclyl, heteroaryl, heterocyclyl, and each of R^{o}, R^{p}, R^{q}, and R^{r}, independently, being H, alkyl, alkenyl, aryl, cyclyl, heteroaryl, heterocyclyl, alkoxyl, nitro, amino, cyano, or halogen.

14. The method of claim 13, wherein each of Rⁿ and R⁵ is alkyl.

15. The method of claim 1, wherein the compound is selected from those listed in Table 1.

16. The method of claim 1, wherein the production of nitric oxide is inhibited by suppressing expression of inducible nitric oxide synthase.

17. A method of inhibiting production of TNFα, comprising administering to a subject in need thereof an effective amount of a compound of the following formula: 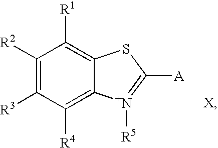
wherein
each of R¹, R², R³, and R⁴, independently, is H, halo, alkyl, alkenyl, akynyl, alkoxyl, aryl, heteroaryl, cylcyl, heterocyclyl, nitro, cyano, amino, amido, or alkoxycarbonyl, or R¹, R², and the two carbon atoms to which they are attached, together form a 3-8 membered ring containing 0, 1, 2, or 3 heteroatoms, or R², R³, and the two carbon atoms to which they are attached, together form a 3-8 membered ring containing 0, 1, 2, or 3 heteroatoms, or R³, R⁴, and the two carbon atoms to which they are attached, together form a 3-8 membered ring containing 0, 1, 2, or 3 heteroatoms;
R⁵ is H, alkyl, aryl, heteroaryl, cyclyl, or heterocyclyl;
A is H, alkyl, aryl, heteroaryl, cyclyl, heterocyclyl, alkoxy, amino, halo, mercapto, thioalkoxy, 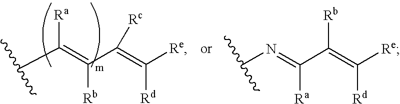
in which m is 0, 1, 2, or 3; each of R^{a}, R^{b}, R^{c}, R^{d}, and R^{e} independently is H, alkyl, akenyl, akynyl, halo, aryl, heteroaryl, cyclyl, heterocylyl, alkoxy, mercapto, thioalkoxy, amino, amido, or R^{d}, R^{e}, and the carbon atom to which they are attached, together form a 3-8 membered ring containing 0, 1, 2, or 3 heteroatoms, which is optionally fused with aryl, heteroaryl, cyclyl, or heterocyclyl;
optionally A, R⁵, the N atom to which R⁵ is attached, and the carbon atom to which A is attached, together form a 4-8 membered ring containing 1, 2, or 3 heteroatoms; and
X is an anion or a negatively charged group attached to an atom of R¹, R², R³, R⁴, R⁵, or A.

18. The method of claim 17, wherein X is I⁻, Br⁻, Cl⁻, ClO₄⁻, C₂H₅SO₄⁻, or 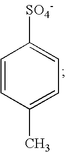
or X is a negatively charged group attached to an atom of R¹, R², R³, R⁴, R⁵, or A, the negatively charged group being selected from —CO₂⁻, —SO₃⁻, or PO₃²⁻.

19. The method of claim 17, wherein A is alkyl.

20. The method of claim 17, wherein R⁵ is alkyl.

21. The method of claim 20, wherein A is

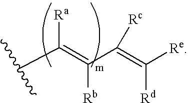

22. The method of claim 21, wherein one of R^{d} and R^{e} is substituted or unsubstituted phenyl.

23. The method of claim 21, wherein one of R^{d} and R^{e} is thioalkyl, amino, or amido.

24. The method of claim 20, wherein R^{d}, R^{e}, and the carbon atom to which they are attached, together form a 3-8 membered ring containing 0, 1, or 2 heteroatoms.

25. The method of claim 24, wherein A is 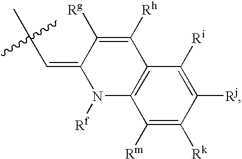
R^{f} being H, alkyl, alkenyl, aryl, cyclyl, heteroaryl, or heterocyclyl; and each of R^{g}, R^{h}, Rⁱ, R^{j}, R^{k}, and R^{m}, independently being H, alkyl, alkenyl, aryl, cyclyl, heteroaryl, heterocyclyl, alkoxyl, halogen, nitro, or cyano.

26. The method of claim 24, wherein A is 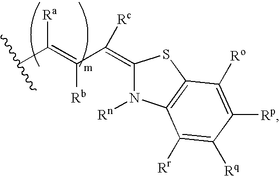
Rⁿ being H, alkyl, alkenyl, aryl, cyclyl, heteroaryl, heterocyclyl, and each of R^{o}, R^{p}, R^{q}, and R^{r}, independently, being H, alkyl, alkenyl, aryl, cyclyl, heteroaryl, heterocyclyl, alkoxyl, nitro, amino, cyano, or halogen.

27. The method of claim 17, wherein the compound is selected from the compounds listed in Table 1.

28. A method of treating coronaviral infection, comprising administering to a subject in need thereof an effective amount of a compound of the following formula: 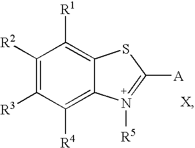
wherein
each of R¹, R², R³, and R⁴, independently, is H, halo, alkyl, alkenyl, akynyl, alkoxyl, aryl, heteroaryl, cylcyl, heterocyclyl, nitro, cyano, amino, amido, or alkoxycarbonyl, or R¹, R², and the two carbon atoms to which they are attached, together form a 3-8 membered ring containing 0, 1, 2, or 3 heteroatoms, or R², R³, and the two carbon atoms to which they are attached, together form a 3-8 membered ring containing 0, 1, 2, or 3 heteroatoms, or R³, R⁴, and the two carbon atoms to which they are attached, together form a 3-8 membered ring containing 0, 1, 2, or 3 heteroatoms;
R⁵ is H, alkyl, aryl, heteroaryl, cyclyl, or heterocyclyl;
A is H, alkyl, aryl, heteroaryl, cyclyl, heterocyclyl, alkoxy, amino, halo, mercapto, thioalkoxy, 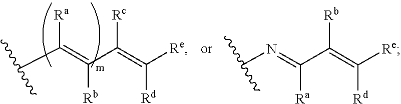
in which m is 0, 1, 2, or 3; each of R^{a}, R^{b}, R^{c}, R^{d}, and R^{e} independently is H, alkyl, akenyl, akynyl, halo, aryl, heteroaryl, cyclyl, heterocylyl, alkoxy, mercapto, thioalkoxy, amino, amido, or R^{d}, R^{e}, and the carbon atom to which they are attached, together form a 3-8 membered ring containing 0, 1, 2, or 3 heteroatoms, which is optionally fused with aryl, heteroaryl, cyclyl, or heterocyclyl;
optionally A, R⁵, the N atom to which R⁵ is attached, and the carbon atom to which A is attached, together form a 4-8 membered ring containing 1, 2, or 3 heteroatoms; and
X is an anion or a negatively charged group attached to an atom of R¹, R², R³, R⁴, R⁵, or A.

29. The method of claim 28, wherein X is I⁻, Br⁻, Cr⁻, ClO₄⁻, C₂H₅SO₄⁻, or 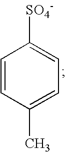
or X is a negatively charged group attached to an atom of R¹, R², R³, R⁴, R⁵, or A, the negatively charged group being selected from —CO₂⁻, —SO₃⁻, or —PO₃²⁻.

30. The method of claim 28, wherein A is alkyl.

31. The method of claim 28, wherein R⁵ is alkyl.

32. The method of claim 31, wherein A is

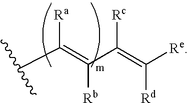

33. The method of claim 32, wherein one of R^{d} and R^{e} is substituted or unsubstituted phenyl.

34. The method of claim 32, wherein one of R^{d} and R^{e} is thioalkyl, amino, or amido.

35. The method of claim 32, wherein R^{d}, R^{e}, and the carbon atom to which they are attached, together form a 3-8 membered ring containing 0, 1, or 2 heteroatoms.

36. The method of claim 35, wherein A is 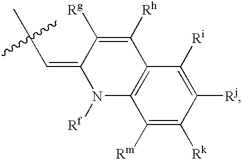
R^{f} being H, alkyl, alkenyl, aryl, cyclyl, heteroaryl, or heterocyclyl; and each of R^{g}, R^{h}, Rⁱ, R^{j}, R^{k}, and R^{m}, independently being H, alkyl, alkenyl, aryl, cyclyl, heteroaryl, heterocyclyl, alkoxyl, halogen, nitro, or cyano.

37. The method of claim 35, wherein A is 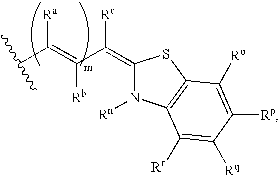
Rⁿ being H, alkyl, alkenyl, aryl, cyclyl, heteroaryl, heterocyclyl, and each of R^{o}, R^{p}, R^{q}, and R^{r}, independently, being H, alkyl, alkenyl, aryl, cyclyl, heteroaryl, heterocyclyl, alkoxyl, nitro, amino, cyano, or halogen.

38. The method of claim 28, wherein the compound is selected from the compounds listed in Table 1.
